(19) United States
(12) Patent Application Publication
(10) Pub. No.: US 2019/0295720 A1
(43) Pub. Date: Sep. 26, 2019

(54) IMMUNE CELL SIGNATURES

(71) Applicant: NantOmics, LLC, Culver City, CA (US)

(72) Inventor: Christopher W. SZETO, Culver City, CA (US)

(21) Appl. No.: 16/358,576

(22) Filed: Mar. 19, 2019

Related U.S. Application Data
(60) Provisional application No. 62/647,621, filed on Mar. 23, 2018.

(51) Int. Cl. G16H 50/20; C12Q 1/6886; C12Q 1/6827; C12Q 1/686; C12Q 1/6858; G16H 50/30

(52) U.S. Cl. CPC G16H 50/20; C12Q 1/6886; C12Q 1/6827; C12Q 1/686; C12Q 1/6858; C12Q 2600/106; C12Q 2600/16; C12Q 2600/158; C12Q 2600/156; C12Q 2600/112; G16H 50/30

(57) ABSTRACT

An immune gene expression signature is associated with clinical features in tumor samples and can be used to predict the immunological state of a tumor and/or sensitivity of the tumor to immune therapy.

This application claims priority to our copending US provisional patent application with the Ser. No. 62/647,621, which was filed Mar. 23, 2018.

### FIELD OF THE INVENTION

The field of the invention is genetic analysis of tumor tissue, especially as it relates to immune cells signatures.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Studies of the tumor microenvironment have surfaced promising avenues of exploration to better understand the clinical relevance of T cell immune biology. Regulatory T cells (Tregs) have keenly emerged in light of their ability to inhibit the adaptive immune response and provide a mechanism of immune escape for cancer cells within the tumor microenvironment across various cancer types. However, the relatively large number of studies exploring the clinical relevance of intratumoral Treg abundance has produced controversial results to date, with some studies finding a poor prognosis associated with Treg infiltration, and others suggesting a favorable Treg-associated prognosis. Not surprisingly, the recent efforts to account for these polarized clinical results have undermined the notion that FOXP3+ Tregs invariably suppress tumor immunity. To address this uncertainty, multiple gene markers were taken into account to more accurately identify Tregs, such as FOXP3+BLIMP1 or FOXP3+CTLA4. However, none of the known studies have produced results that were suitable to guide a clinician towards a rational-based therapy with high confidence in a predicted outcome.

Indeed, immune heterogeneity within the tumor microenvironment has added multiple layers of complexity to the understanding of chemosensitivity and survival across various cancer types. Within the tumor microenvironment, immunogenicity is a favorable clinical feature in part driven by the antitumor activity of CD8+ T cells. However, tumors often inhibit this antitumor activity by exploiting the suppressive function of Regulatory T cells (Tregs), thus suppressing an adaptive immune response.

Unfortunately, there are numerous mechanisms other than Tregs and CD8+ T involved in the immunogenicity of tumor cells, and an accurate prediction of immunogenicity of a tumor has remained elusive. Indeed, it has been reported that the immune infiltrate composition changes at each tumor stage and that particular immune cells have a major impact on survival. For example, densities of T follicular helper (Tfh) cells and innate cells increases, and most T cell densities decrease where tumor progression is observed. Moreover, the number of B cells, which are key players in the core immune network and are associated with prolonged survival, increase at a late stage and often show a dual effect on recurrence and tumor progression (see e.g., Immunity 2013 Oct 17;39(4):782-95).

Therefore, despite numerous findings in isolation, complex interactions between tumors and their microenvironment remain to be elucidated. Consequently, there is still a need for improved systems and methods to better characterize immunogenicity of a tumor.

### SUMMARY OF THE INVENTION

The inventive subject matter is directed to various methods of genetic analysis, and especially quantitative and normalized RNA expression analysis of tumor tissue, to thereby allow for identification of infiltration and/or activity of various immune cells in a specific tumor. For example, in some embodiments, the inventors used various gene sets associated with various immune cells types and then correlated them with specific disease categories (e.g., ICD10 categories) to predict whether or not a tumor is immune-enriched. Moreover, immune cell-enrichment was found to be correlated with PDL1 high/normal/low cases, and molecular targets could also be identified for patients where PDL1 is low.

In one aspect of the inventive subject matter, the inventors contemplate a method of characterizing a tumor that includes a step of quantifying or obtaining expression levels for a plurality of distinct genes, wherein the distinct genes are associated with (e.g., expressed in, most typically specifically expressed in) respective distinct types of immune cells, and a further step of determining over-expression or under-expression for each of the distinct genes relative to respective reference ranges, wherein the reference ranges are specific for a specific tumor type. In yet another step the over-expression and/or under-expression of each of the distinct genes is then used to infer activity and/or infiltration by the immune cells in the tumor.

Most typically, but not necessarily, the expression level is measured via qPCR or RNAseq, and suitable genes for such analysis include BLK, CD19, CR2 (CD21), HLA-DOB, MS4A 1 (CD20), TNFRSF17 (CD269), CD2,CD3E,CD3G,CD6, ANP32B (APRIL), BATF, NUP107, CD28, ICOS (CD278), CD38, CSF2 (GM-CSF), IFNG, IL12RB2,LTA, CTLA4 (CD152), TXB21, STAT4, CXCR6 (CD186), GATA3, IL26, LAIR2 (CD306), PMCH, SMAD2, STATE, IL 17A, IL 17RA (CD217), RORC, CXCL13, MAF, PDCD1 (CD279), BCL6, FOXP3, ATM, DOCKS, NEFL, REPS1, USP9Y, AKT3, CCR2 (CD192), EWSR1 (EWS), LTK, NFATC4, CD8A, CD8B, FLT3LG, GZMM, MET1, PRF1, CD160, FEZ1, TARP (TCRG), BCL2, FUT5, NCR1 (CD335), ZNF205, FOXJ1, MPPED1, PLA2G6, RRAD, GTF3C1, GZMB, IL21R (CD360), CCL13, CCL17, CCL22 (MDC), CD209, HSD11B1, CD1A, CD1B, CD1E, F13A1, SYT17, CCL1, EBI3, IDO1 (INDO), LAMP3 (CD208), OAS3, IL3RA (CD123), APOE, CCL 7 (FIC), CD68, CHIT1, CXCL5, MARCO, MSR1 (CD204), CMA1, CTSG, KIT (CD117), MS4A2, PRG2, TPSAB1, CSF3R (CD114), FPR2, MME (CD10), CCR3 (CD193), IL5RA (CD125), PTGDR2, (CD294), SMPD3, and THBS1.

In further contemplated embodiments, a threshold for determination of over-expression or under-expression may be when the quantified expression level exceeds +/−2SD of the reference range. Most preferably, the reference range is specific for a particular tumor type as classified in ICD10. As will be readily appreciated, the immune status may then be associated with the tumor based on the inferred activity and/or infiltration. Consequently, immune therapy such as treatment with a checkpoint inhibitor, treatment with immune stimulatory compositions, and/or vaccination with a tumor associated antigen or tumor and patient specific may then be recommended or initiated. For example, checkpoint inhibitor treatment with a PDL1 inhibitor may be used for a PDL1-high tumor, while checkpoint inhibitor treatment with a TIM3 inhibitor or an IDO inhibitor may be recommended or initiated for a PDL1-low tumor.

Therefore, viewed from a different perspective, the inventor also contemplates a method of identifying a patient for immune therapy that will include a step of quantifying or obtaining expression levels for a plurality of distinct genes, wherein the distinct genes are associated with respective distinct types of immune cells. In a further step, over-expression or under-expression is determined for each of the distinct genes relative to respective reference ranges, wherein the reference ranges are specific for a specific tumor type, and in yet another step, the over-expression and/or under-expression of each of the distinct genes is used to infer activity and/or infiltration by the immune cells in the tumor. The so inferred activity is then used to predict an increased likelihood of positive treatment outcome where the inferred activity and/or infiltration of distinct immune cells in the tumor is increased relative to the respective reference ranges, and the patient is selected or identified as a suitable candidate for immune therapy upon prediction of the increased likelihood.

For example, the distinct immune cells in the tumor include pDC, aDC, TFH, NK cells, neutrophils, Treg, iDC, macrophages,Thelper cells, NK cells, CD8 T cells, T cells, and Th1 cells, and/or the increased number may be with respect to at least three or four distinct types of immune cells in the tumor. Suitable genes for such analysis include those noted above, and over-expression or under-expression may be ascertained when the quantified expression level exceeds +/−2SD of the reference range. As will be readily appreciated, suitable immune therapies include treatment with a checkpoint inhibitor, a vaccine composition, and/or an immune stimulatory cytokine.

Therefore, the inventor also contemplates the use of a plurality of distinct genes to characterize a tumor or to predict treatment outcome for immune therapy of the tumor, wherein the plurality of distinct genes are associated with respective distinct types of immune cells, and wherein the use comprises a quantification of expression levels of the distinct genes. Once more, suitable genes for such analysis include those noted above, and over-expression or under-expression for each of the distinct genes is preferably determined relative to respective reference ranges, wherein the reference ranges are specific for a specific tumor type. Thus, methods contemplated herein may also be used to characterize a tumor as being immunologically ‘hot’ or ‘cold’.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is an exemplary flowchart of a method according to the inventive subject matter.

FIG. 2 depicts RNAseq expression of genes in the immune cell panel of FIG. 1 in 1037 clinical cases.

FIG. 3 exemplarily depicts immune cell category activation stratified by tissue-type of the tumor.

FIGS. 4A-4H illustrates exemplary immune cell infiltration/activation for specific immune cell types stratified by tissue-type of the tumor.

FIG. 5 is a table listing statistics for each cancer type.

FIG. 6 is an exemplary report showing high/normal/low calls for a specific tumor sample with regard to ICD10, and z-scores, with detailed results provided for each cell type.

FIG. 7 shows exemplary checkpoint expression patterns for various immune related genes stratified by PDL1 expression category.

FIG. 8 depicts exemplary immune-cell activation in PDL1 categories, allowing for a determination as to whether tissue samples are enriched or suppressed in those cell types.

FIG. 9 depicts associations between immune cell presence/activation in tumor cells as a further function of CMS type, MSI status, and sidedness as determined using the methods presented herein.

FIG. 10 shows exemplary results for immune cell enrichment in MSI and MSS groups as determined using the methods presented herein.

FIG. 11 shows exemplary results for various immune markers MSI high and low groups.

### DETAILED DESCRIPTION

The inventor has discovered that immune cell signatures can be obtained from a tumor tissue using gene expression signatures that are specific to or at least characteristic for various immune cells. Viewed from a different perspective the inventors conducted single-cell experiments to define gene sets that can differentiate between immune-cell types. By observing expression patterns of those gene sets within a tumor sample, the inventor was then able to make a determination as to whether a tumor tissue sample is enriched or suppressed in those cell types.

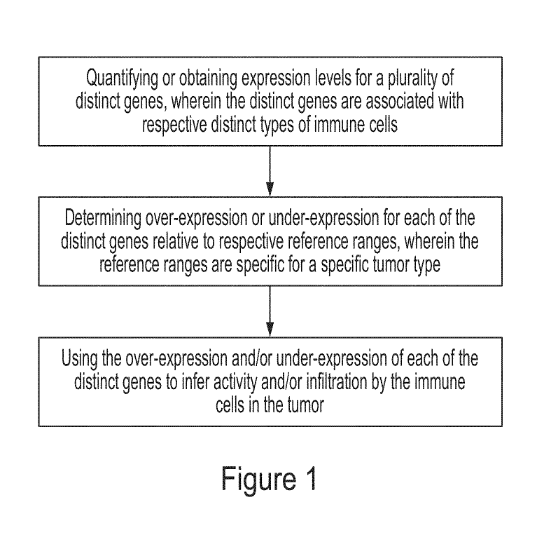

More specifically, based on single cell gene expression analysis of various immune cells, the inventor identified the following genes as being suitable for use in the analyses presented herein: BLK, CD19, CR2 (CD21 ), HLA-DOB, MS4A 1 (CD20), TNFRSF17 (CD269), which are commonly associated with B cells and are involved in several roles, including generating and presenting antibodies, cytokine, production, and lymphoid tissue organization, CD2,CD3E,CD3G,CD6, which are commonly associated with T cells, various genes associated with helper T cells, including ANP32B (APRIL), BATF, NUP107, CD28, ICOS (CD278) (associated with effector T cells), CD38, CSF2 (GM-CSF), IFNG, IL12B2, LTA, CTLA4 (CD152), TXB21, STAT4 (associated with T_{H}1 cells), CXCR6 (CD186), GATA3, IL26, LAIR2 (CD306), PMCH, SMAD2, STATE (associated with T_{H}2 cells), IL 17A, IL 17RA (CD217), RORC (associated with T_{H}17 cells), CXCL13, MAF, PDCD1 (CD279), BCL6 (associated with T_{FH} cells), FOXP3 (associated with T_{reg} cells), ATM, DOCKS, NEFL, REPS1, USP9Y (associated with T_{CM} cells), AKT3, CCR2 (CD192), EWSR1 (EWS), LTK, NFATC4 (associated with T_{EM} cells), CD8A, CD8B, FLT3LG, GZMM, MET1, PRF1 (associated with CD8+ T cells), CD160, FEZ1, TARP (TCRG) (associated with T_{γδ} cells), BCL2, FUT5, NCR1 (CD335), ZNF205 (associated with NK cells), FOXJ1, MPPED1, PLA2G6, RRAD (associated with CD56_{bright} cells), GTF3C1, GZMB, IL21R (CD360) (associated with CD56_{dim} cells), CCL13, CCL17, CCL22 (MDC), CD209, HSD11B1 (associated with dendritic cells), CD1A, CD1B, CD1E, F13A1, SYT17 (associated with immature dendritic cells), CCL1, EBI3, IDO1 (INDO), LAMP3 (CD208), OAS3 (associated with activated dendritic cells), IL3RA (CD123) (associated with plasmacytoid dendritic cells), APOE, CCL 7 (FIC), CD68, CHIT1, CXCL5, MARCO, MSR1 (CD204) (associated with macrophages), CMA1, CTSG, KIT (CD117), MS4A2, PRG2, TPSAB1 (associated with mast cells), CSF3R (CD114), FPR2, MME (CD10) (associated with neutrophils), and CCR3 (CD193), IL5RA (CD125), PTGDR2, (CD294), SMPD3, and THB S1 (associated with eosinophils). These genes were identified to be preferentially or even selectively expressed in certain immune cells (see also e.g., Immunity 39, 782-795, Oct. 17, 2013). FIG. 1 depicts an exemplary flowchart of a method contemplated herein.

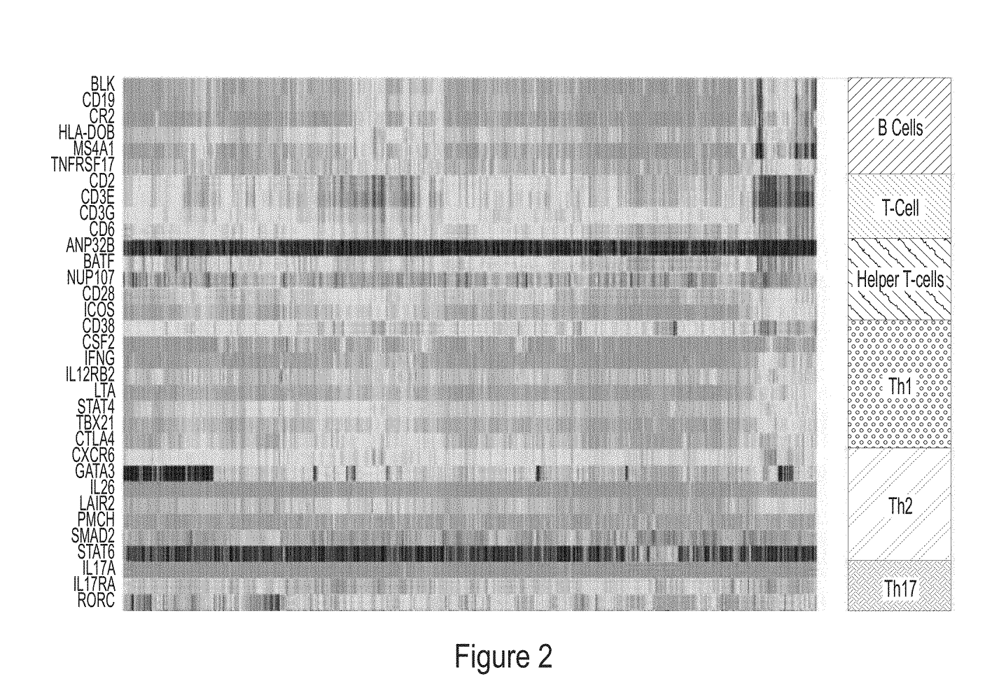

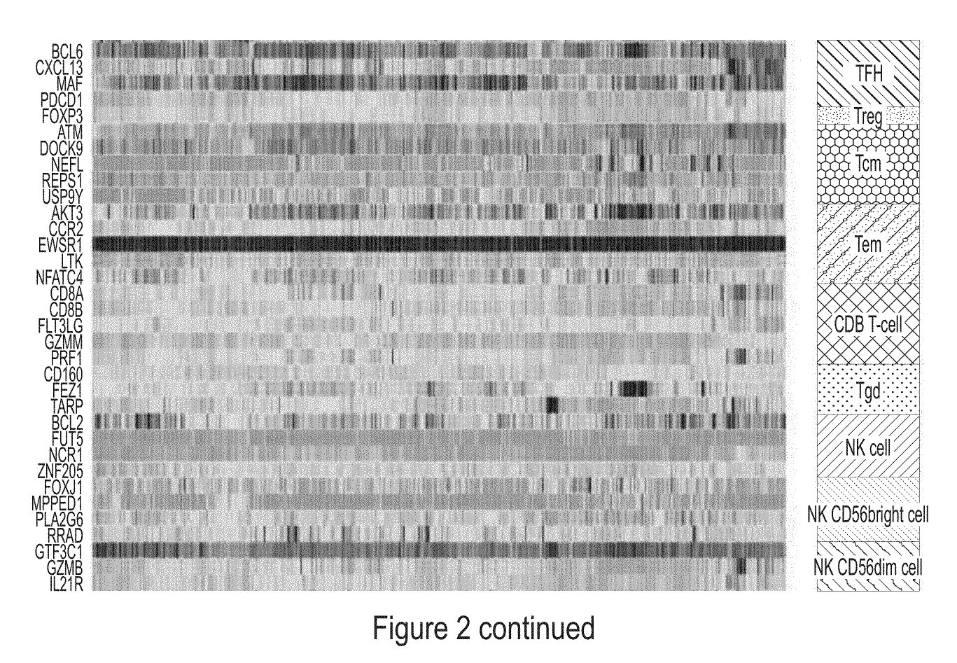

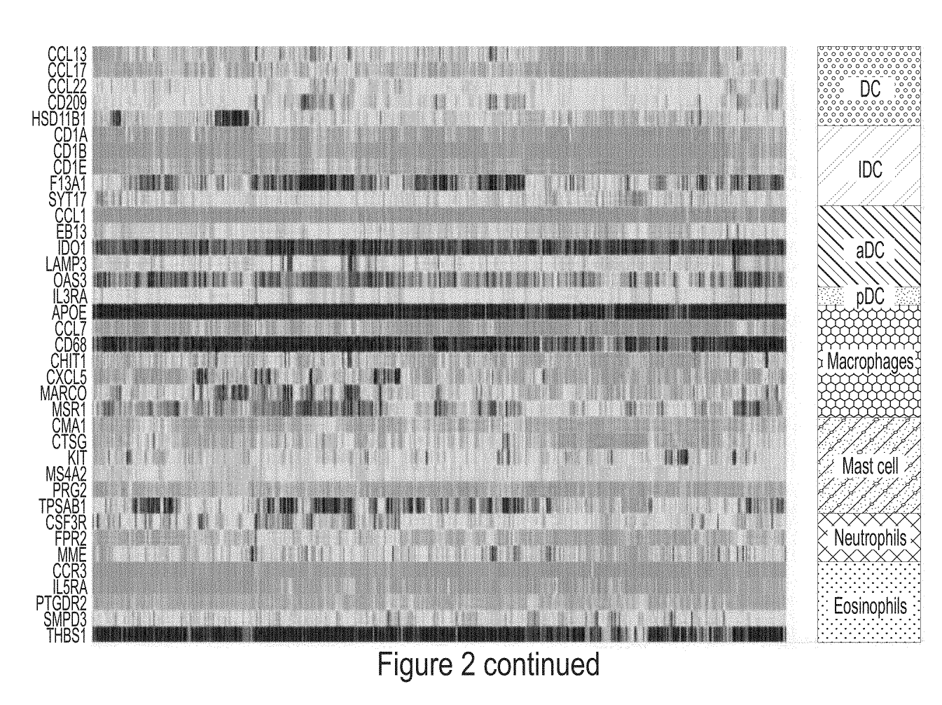

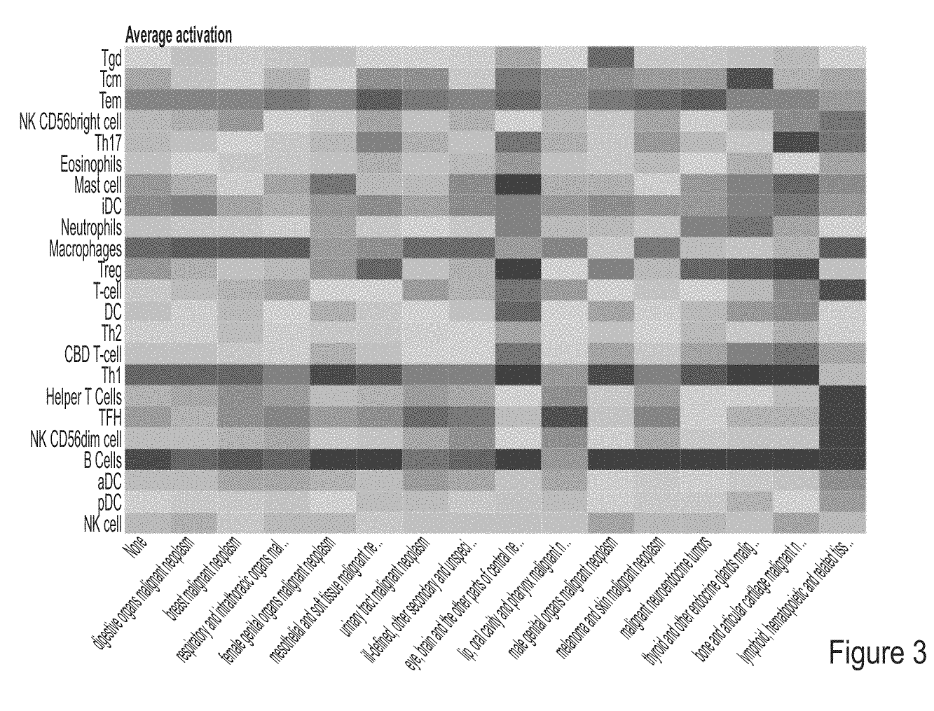

Using these so identified genes, RNAseq analysis was performed on a total of 1037 tumor samples to investigate whether RNA expression levels of these genes would cluster. FIG. 2 depicts an exemplary result for these tumor samples where the rows are ordered by immune cell categories per FIG. 1, and where the columns are ordered by hierarchical clustering using Pearson similarity score. Colors range from blue (log2[TPM+1]==0) to red (log2[TPM+1]˜12.5). Notably, when expression of the immune genes for each immune cell type was averaged, and when the average values were correlated with different cancer types, specific signatures became apparent as is exemplarily illustrated in FIG. 3. Here, the heat map shows an average expression for all genes in each immune cell category, split up into reported ICD10 categories (which are representative of tumor classifications). The rows are ordered by hierarchical clustering (using Pearson similarity score), while the columns are ordered from left-to-right by how many samples were annotated for that cancer type. Colors range from blue (avg. log2[TPM+1]˜0.35) to red (avg. log2[TPM+1]˜5.0).

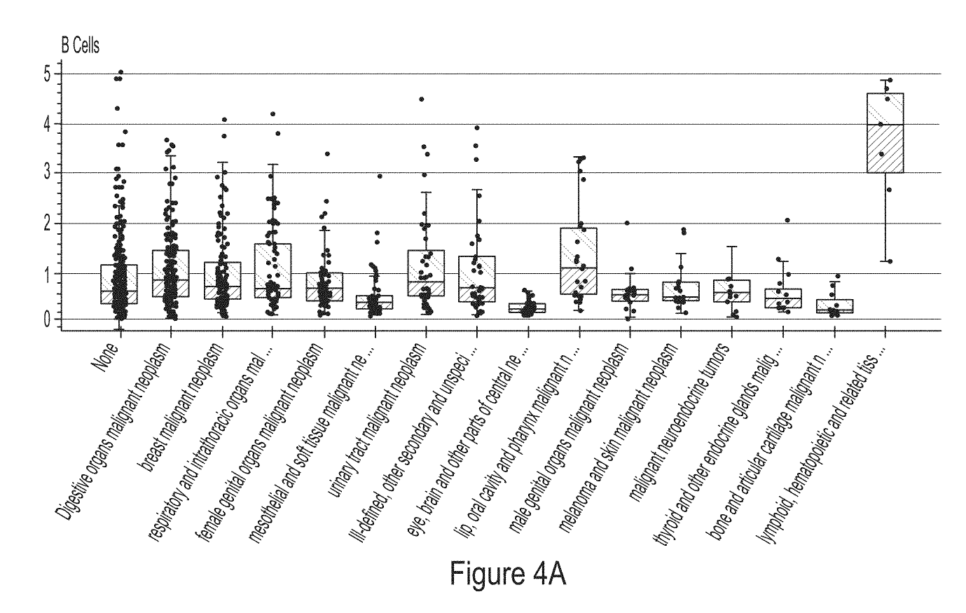

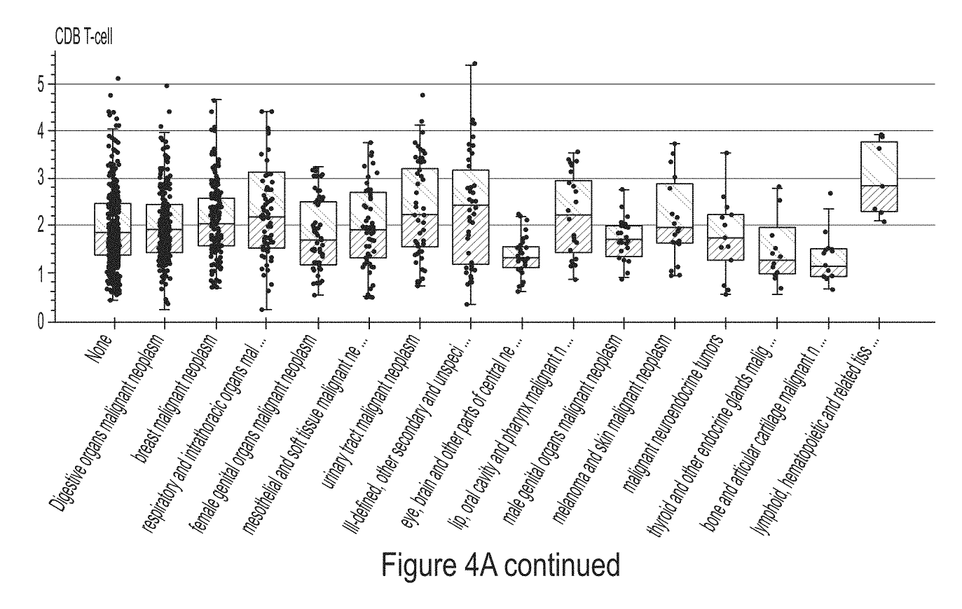

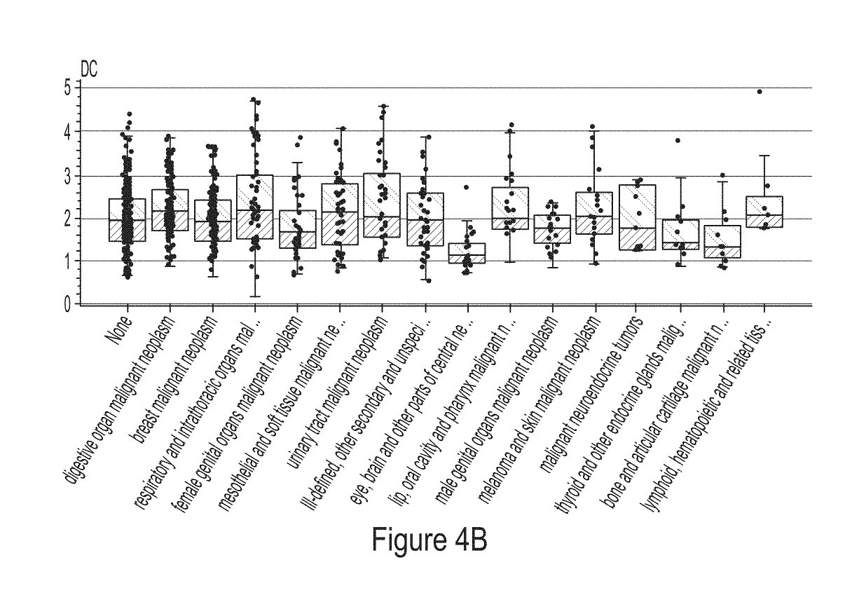

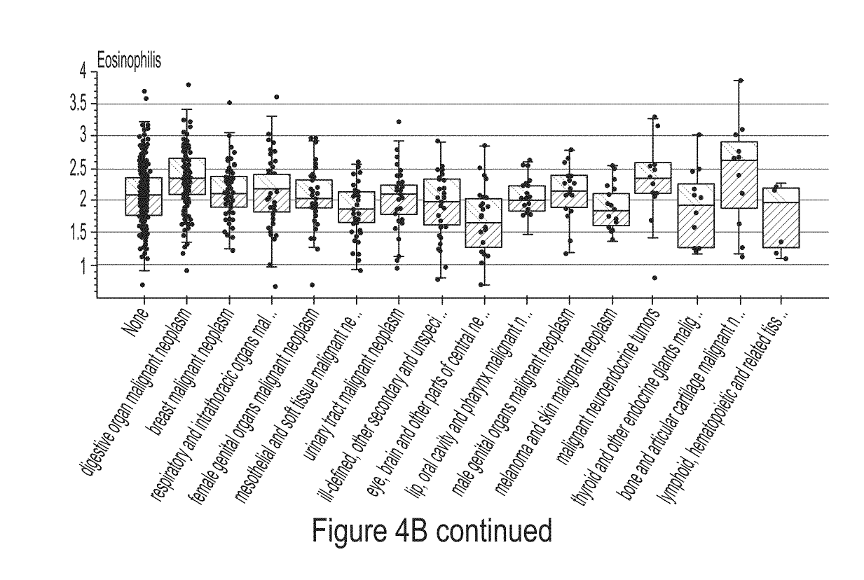

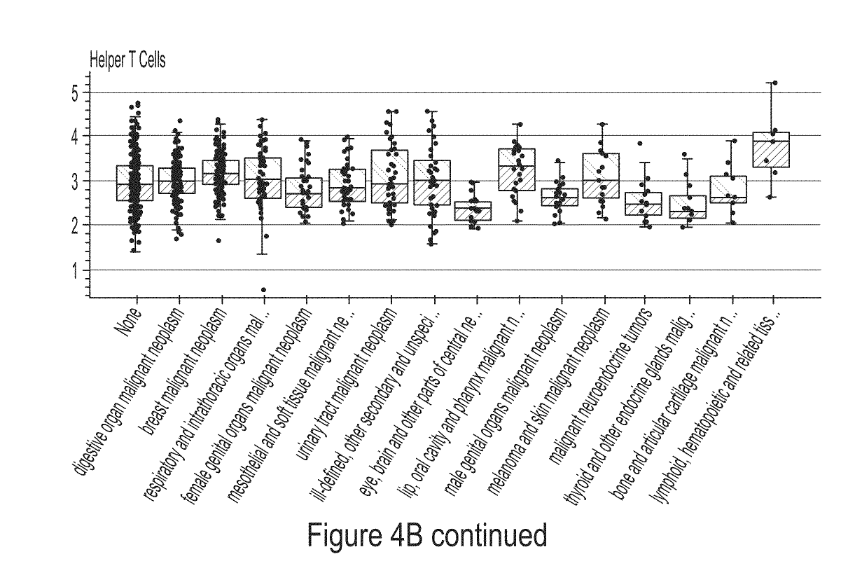

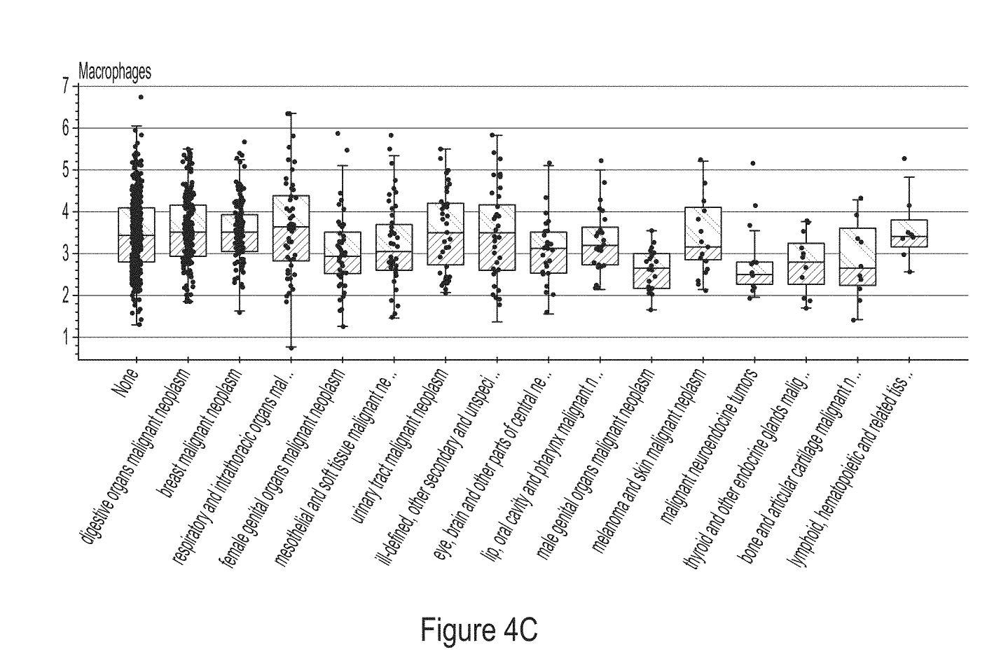

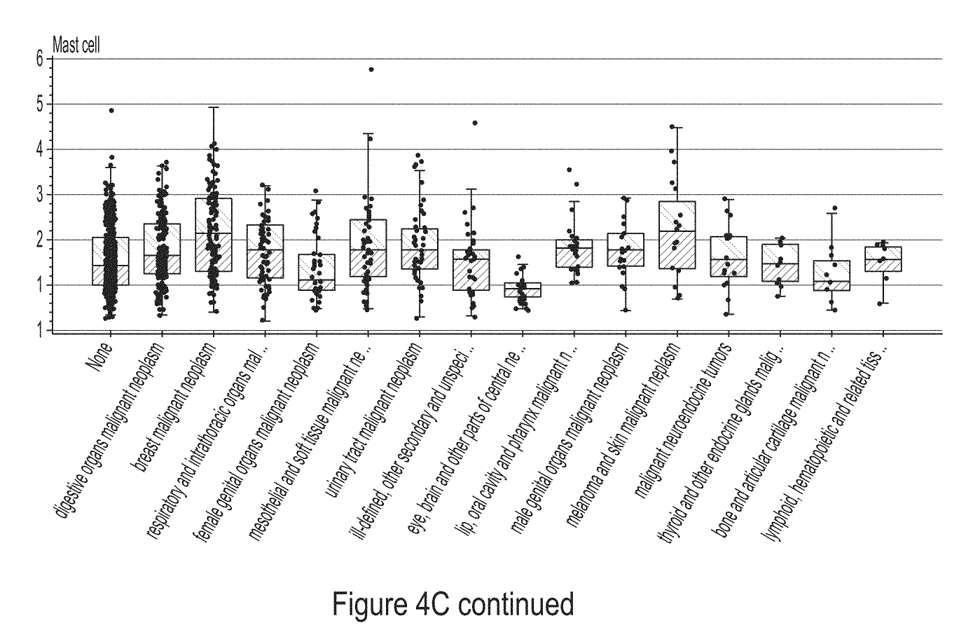

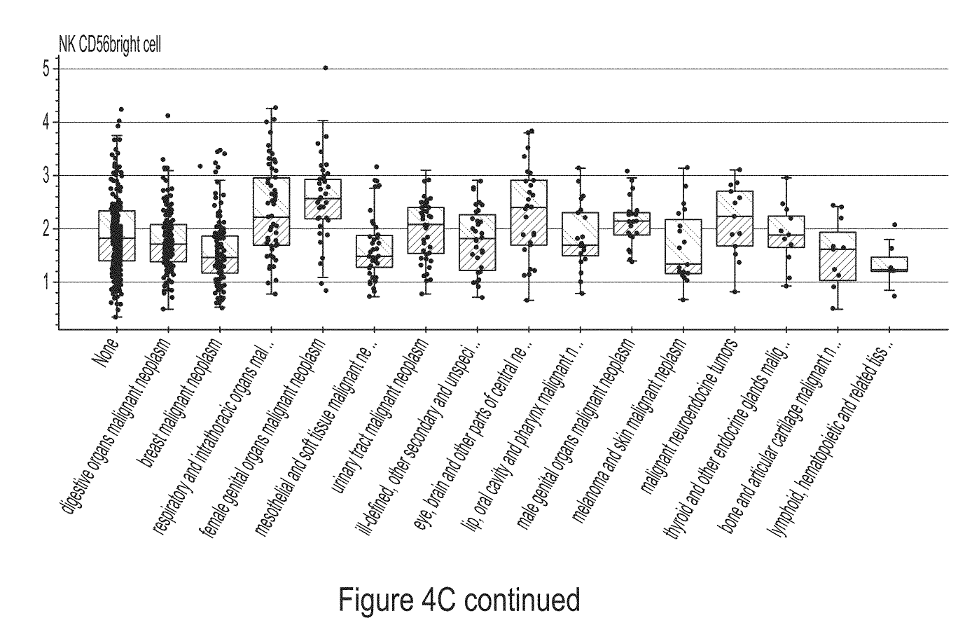

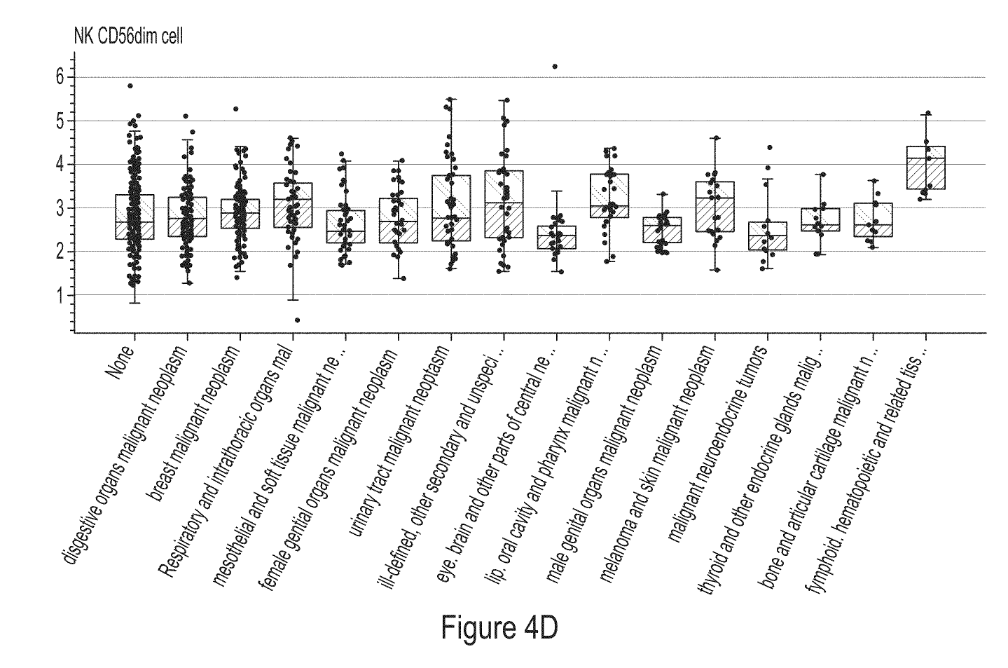

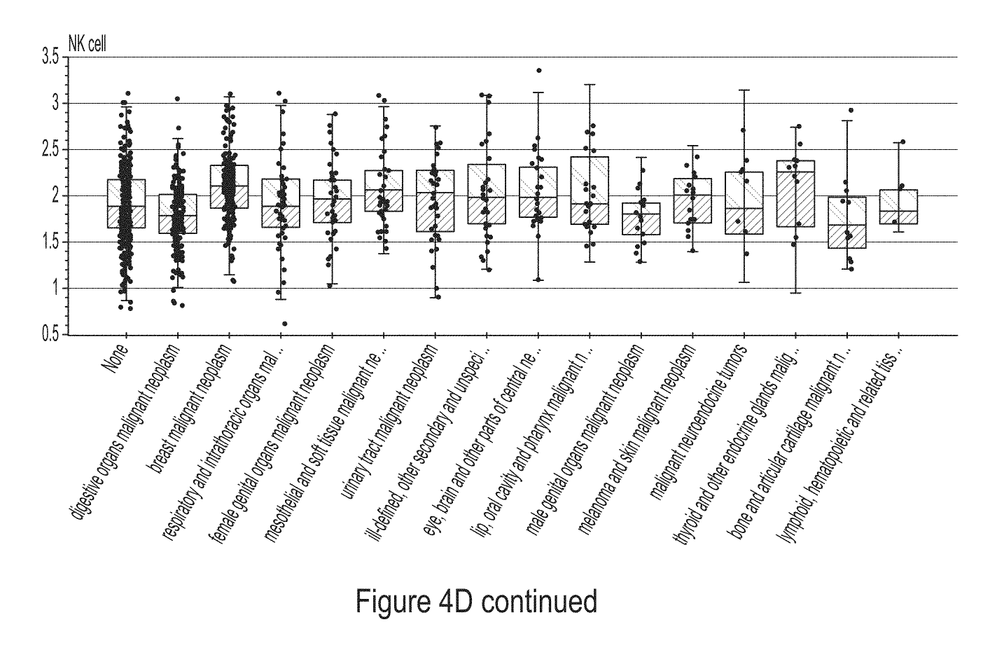

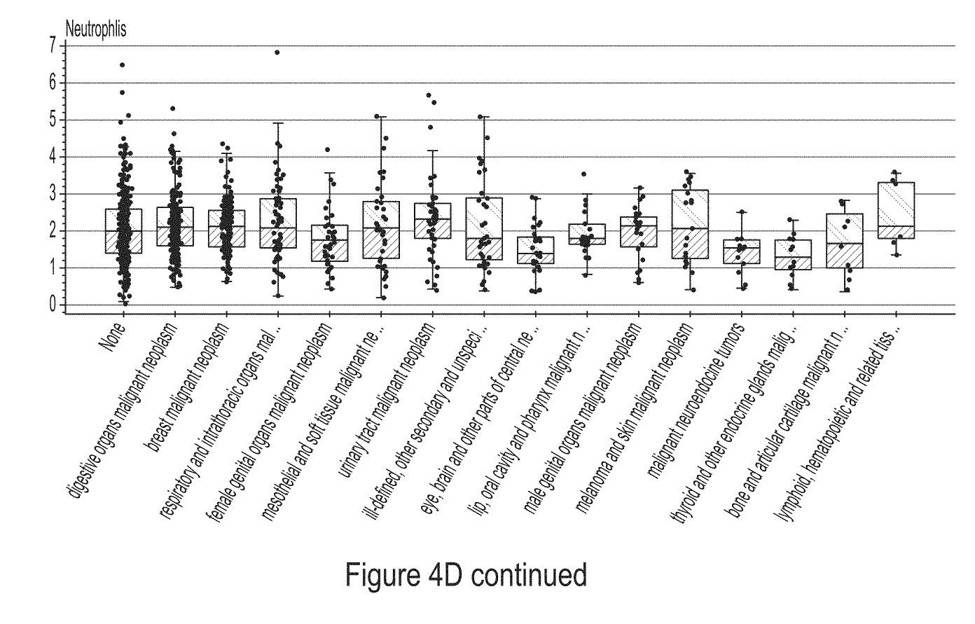

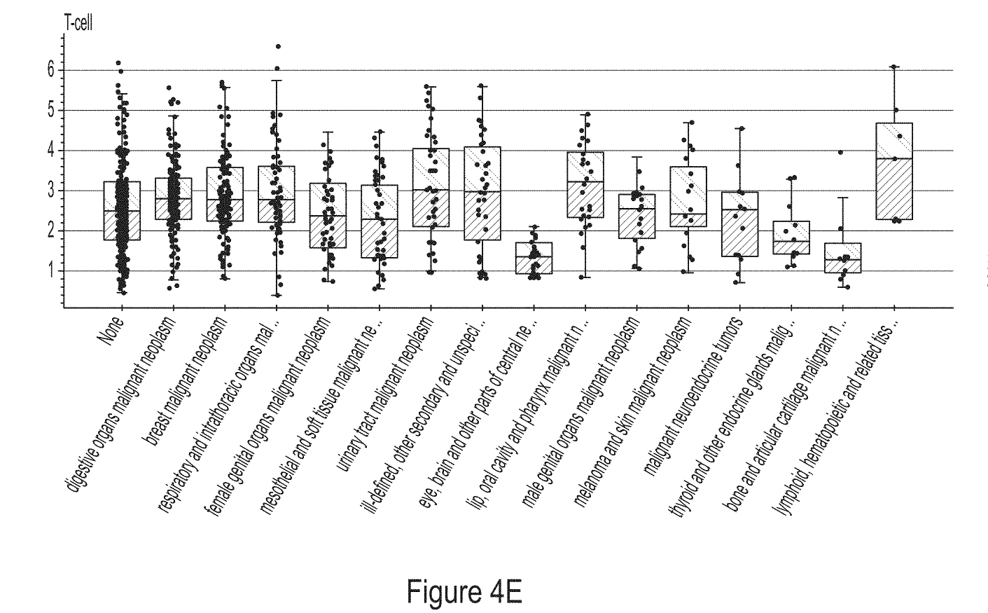

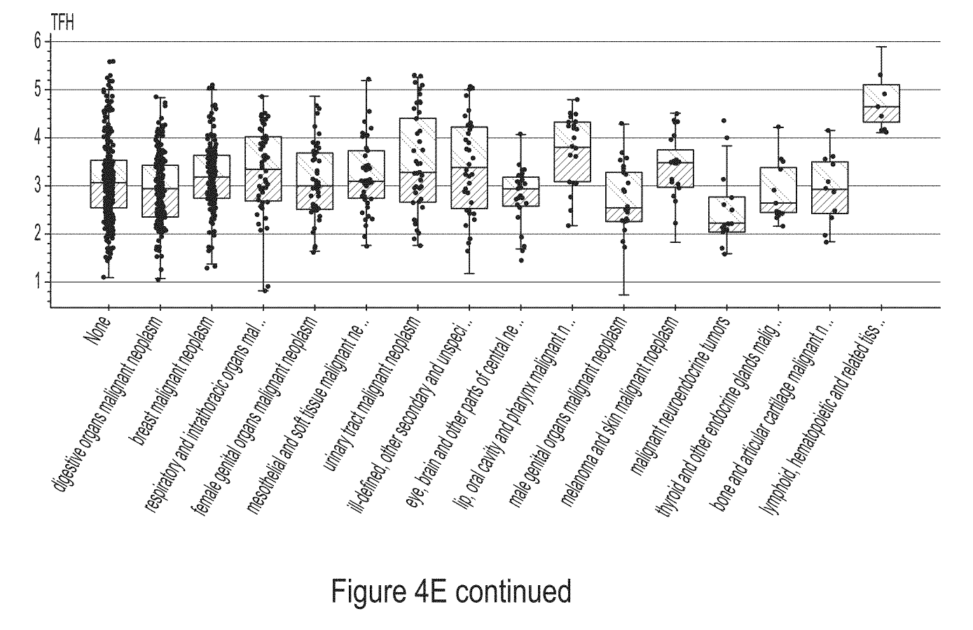

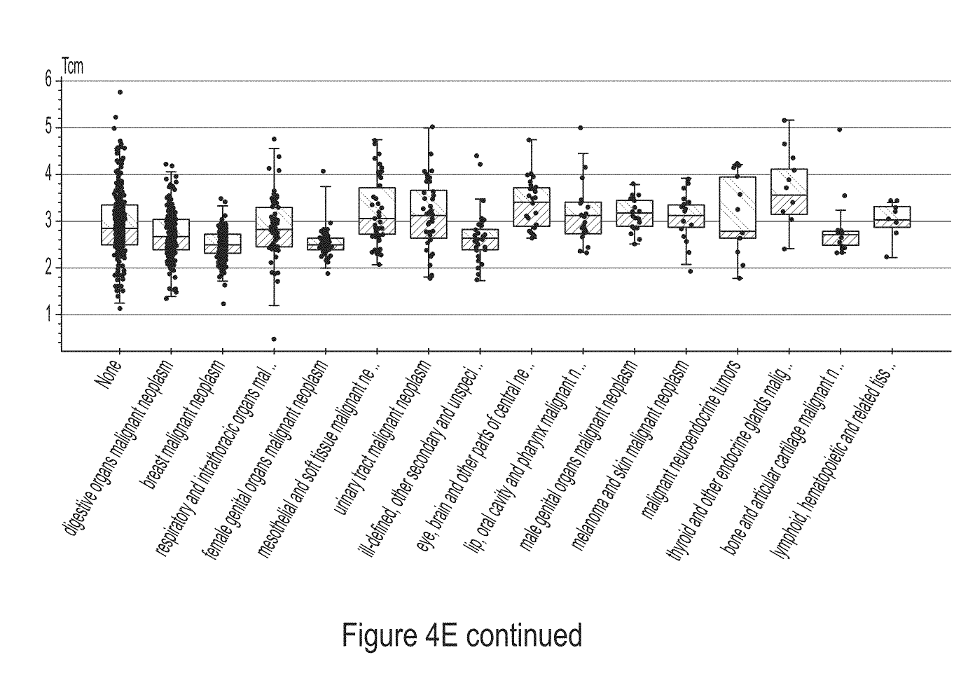

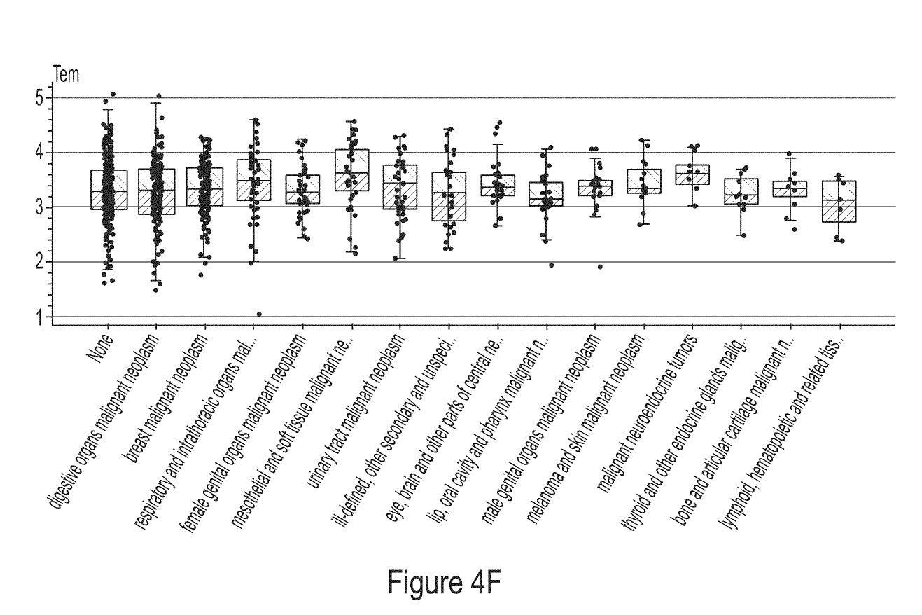

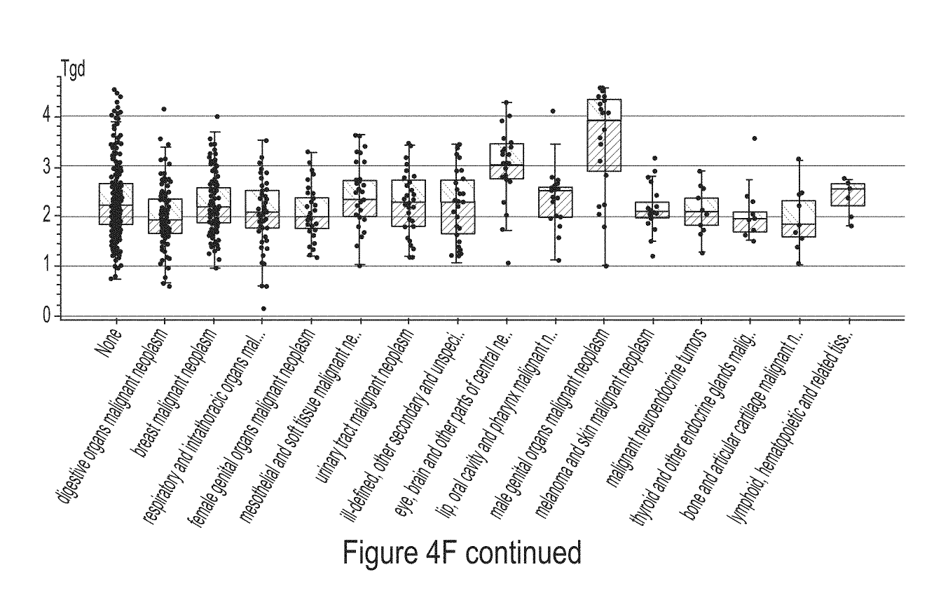

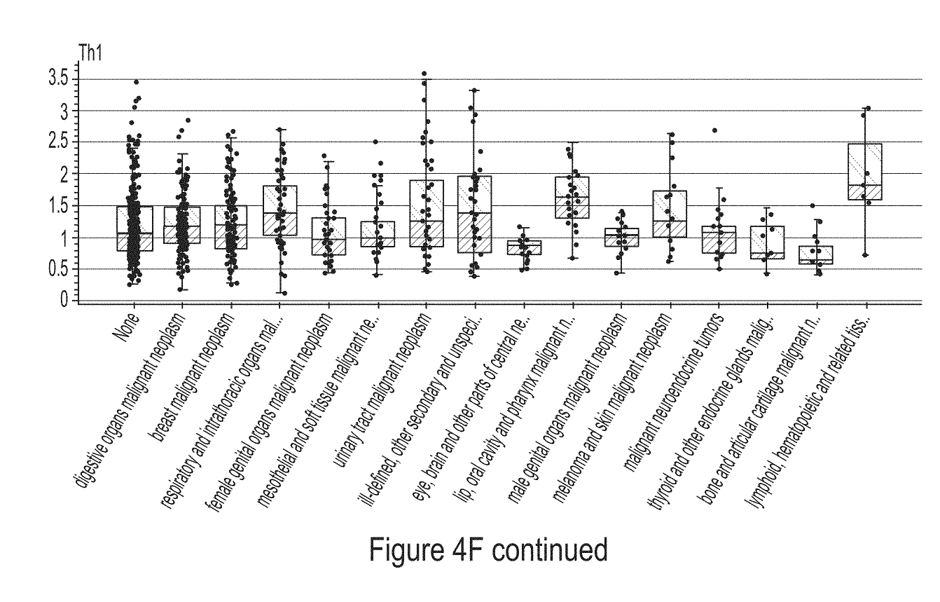

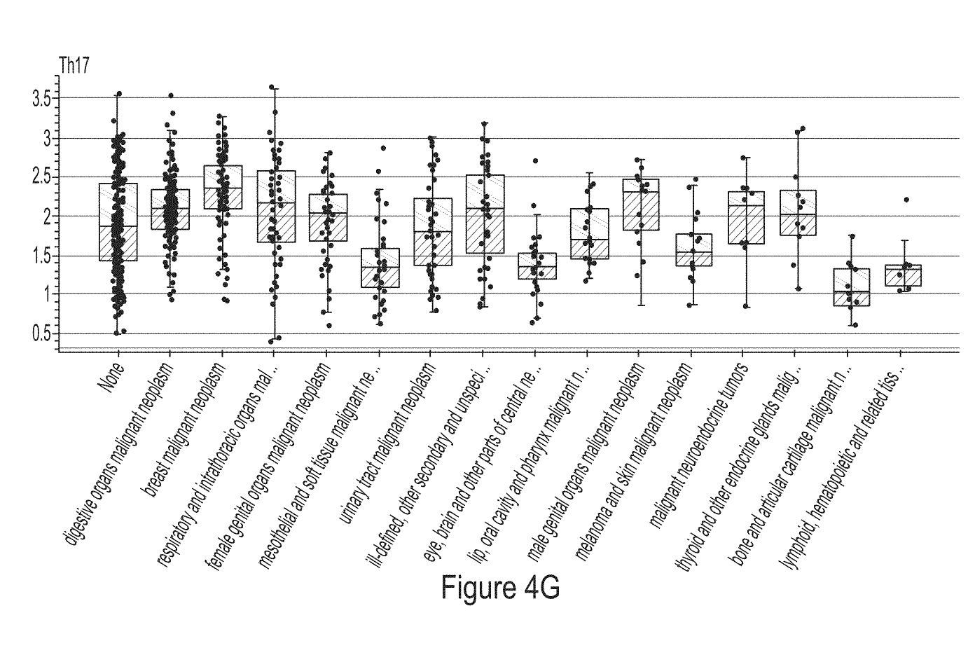

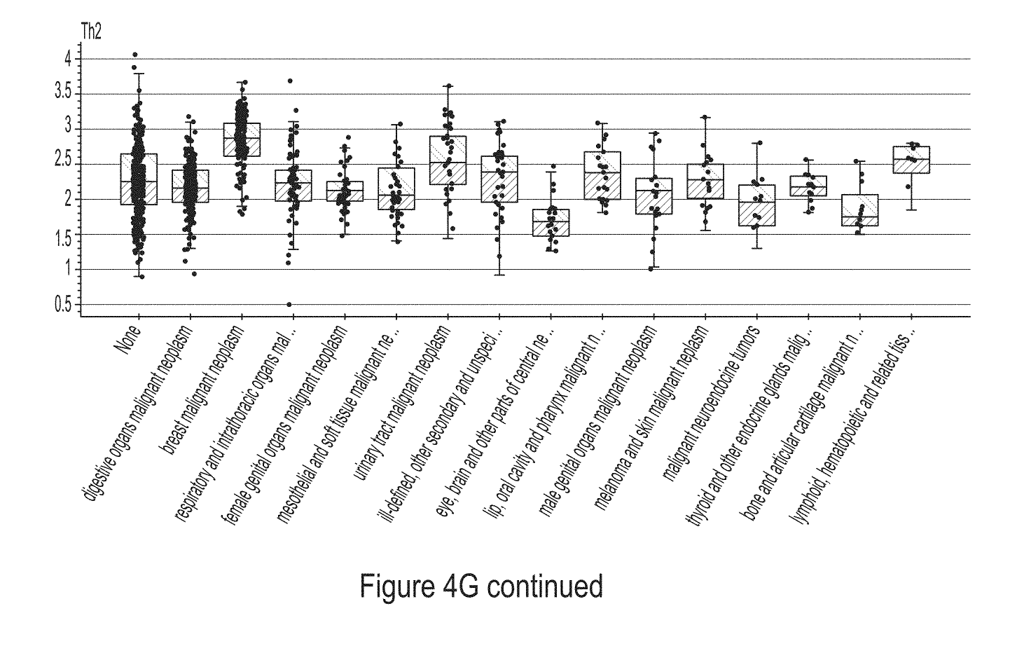

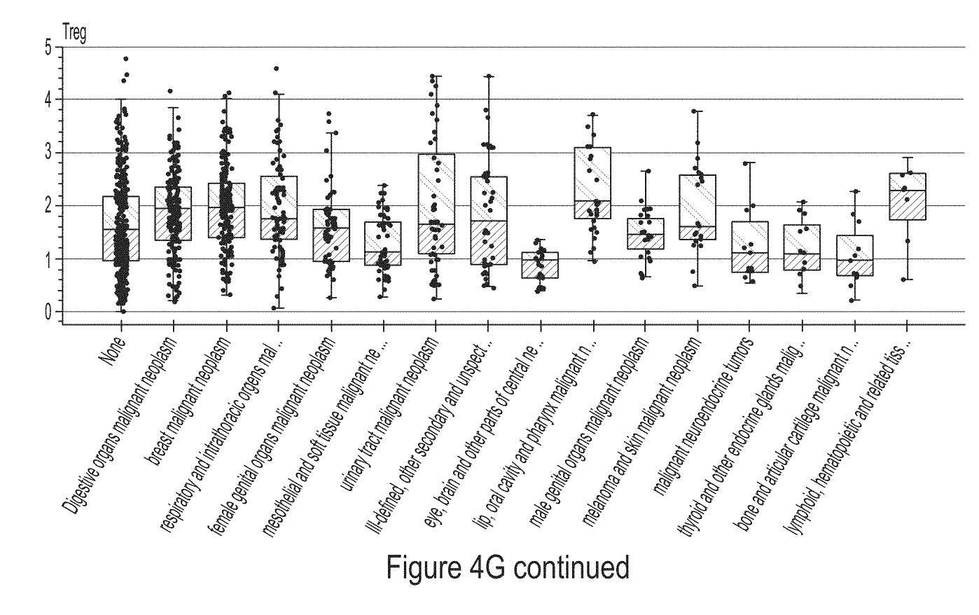

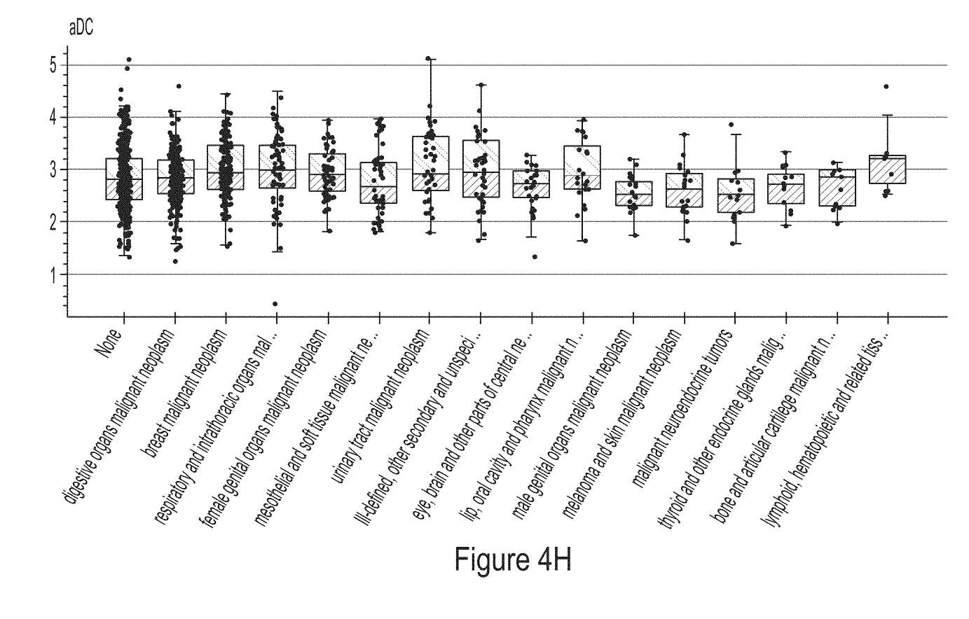

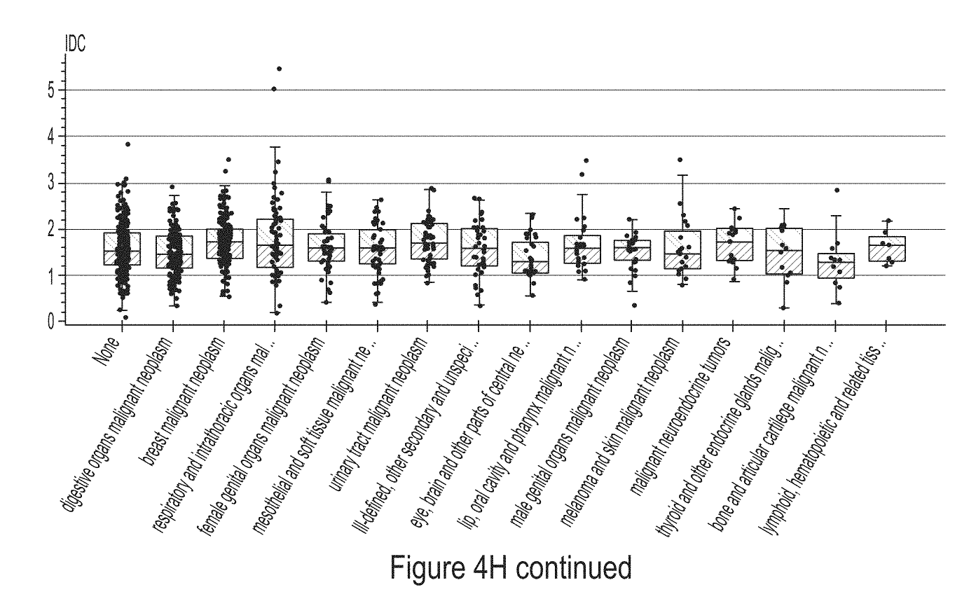

The inventor then investigated whether specific immune cell types would be equally or differentially present or active in different types of tumors. Unexpectedly, as can be seen from the graphs in FIGS. 4A-411, distinct activation patters became evident for the particular immune cell type and cancer involved. More specifically, all RNA expression data were analyzed using log2[tpm+1] expression for all genes in each immune cell category and split up into reported ICD10 categories. The data points in FIGS. 4A-4H are individual reported cases, boxplots are derived from the category (max z=1.5), and the cancer type categories are ordered from left-to-right by how many samples were annotated for that cancer type. As is readily apparent from the results, the strength of expression for the same genes of a single immune cell type varied significantly among different tumor cell types. Moreover, to a lesser degree the range of expression also varied among different tumor cell types. It should further be appreciated that the diversity in gene expression of a single immune cell type among different tumor types was similarly observed for different immune cell types within the same tumor tissue type. Viewed from a different perspective, gene expression of the above noted genes in immune cells was idiosyncratic with regard to a specific tumor type and type of immune cell.

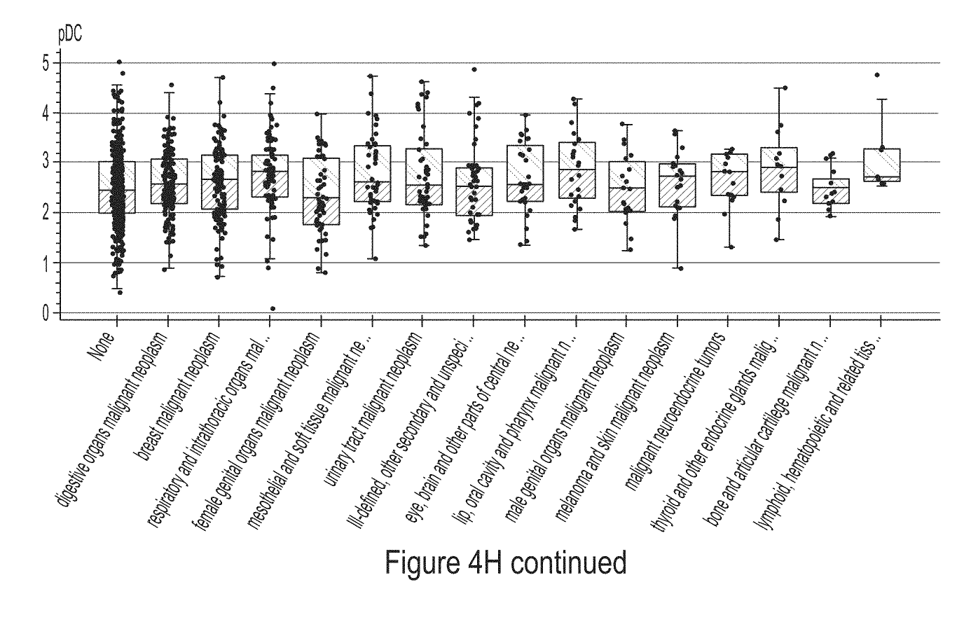

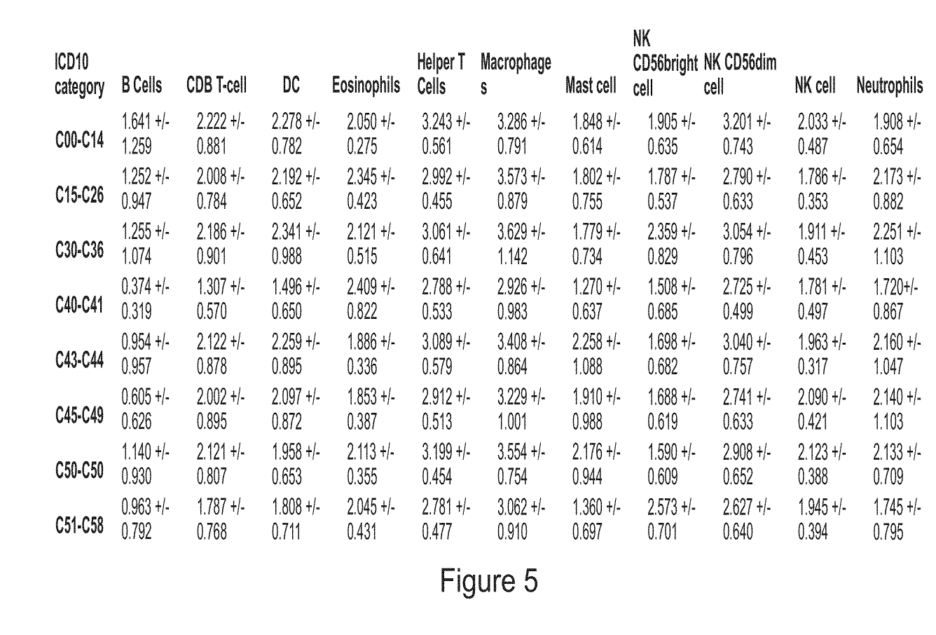

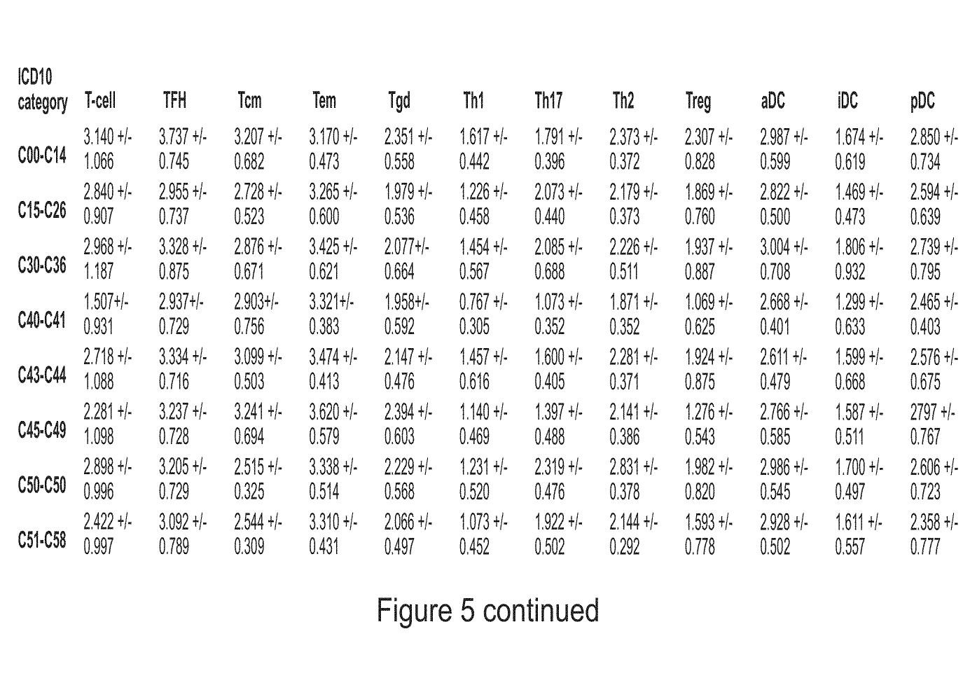

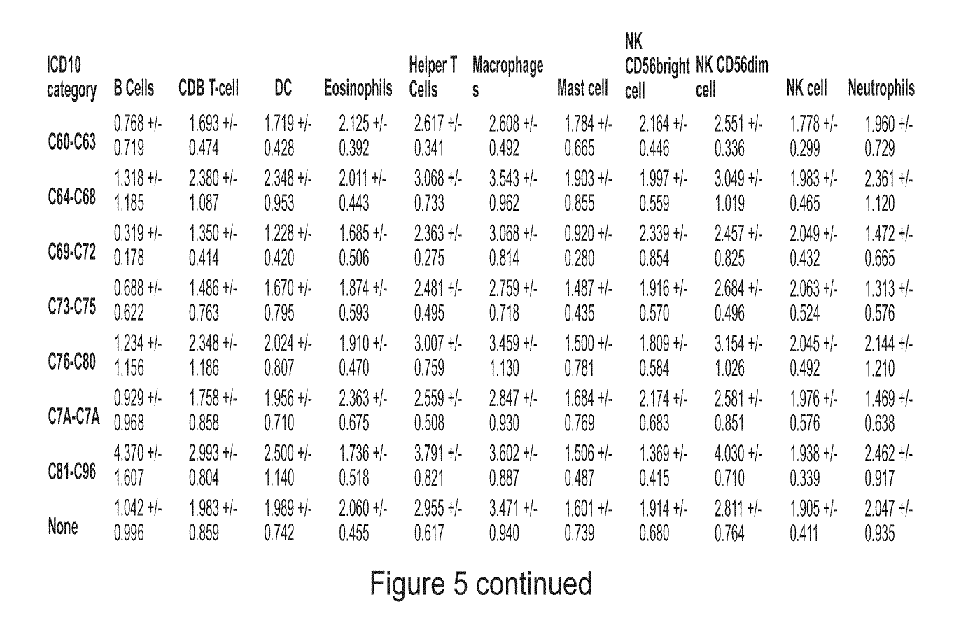

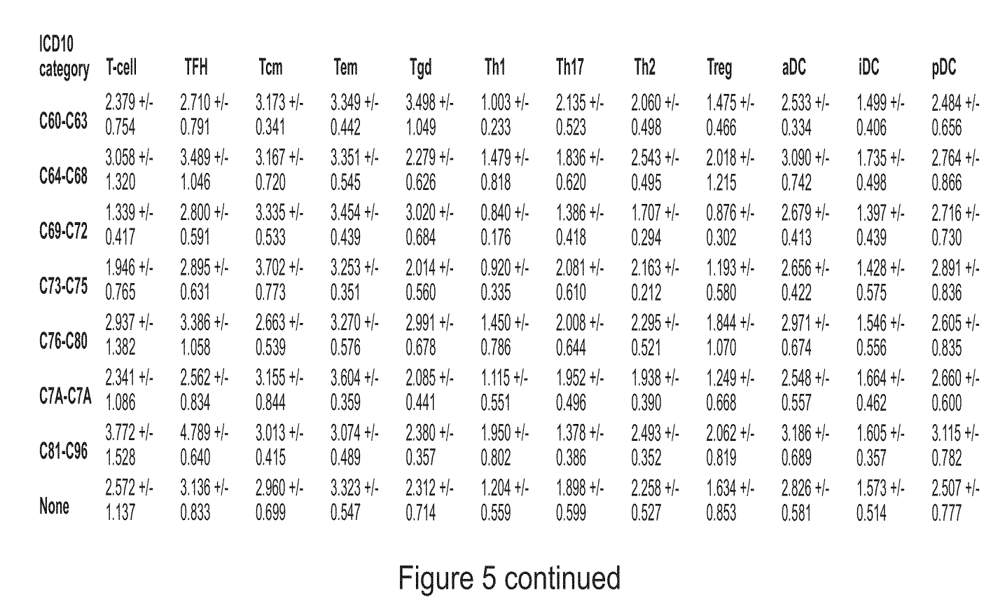

The inventor then employed statistical analysis for the average gene expression of the particular immune cell and cancer type to identify threshold expression levels for the genes in specific immune cells with regard to a specific tumor cell type. Exemplary results are shown in the table of FIG. 5. Here, the mean and standard deviation log2[tpm+1] for all genes in each immune cell category are listed, and stratified into the reported ICD10 category. Once more, it can be readily seen from the data in FIG. 5 that different immune cell categories had different mean expression rates for the genes specified above and in FIG. 1. Consequently, using such deconvoluted information, these statistics can then be advantageously used to determine over-(>2sd), under-(>−2sd), or normal-activation given a particular tumor tissue type. Thus, it should be appreciated that such quantitative analytic process can be used to correlate gene expression (e.g., as measured by RNAseq) with the presence and/or activity of specific immune cells in the tumor, and with that to infer whether a tumor is immunologically ‘hot’ or ‘cold’.

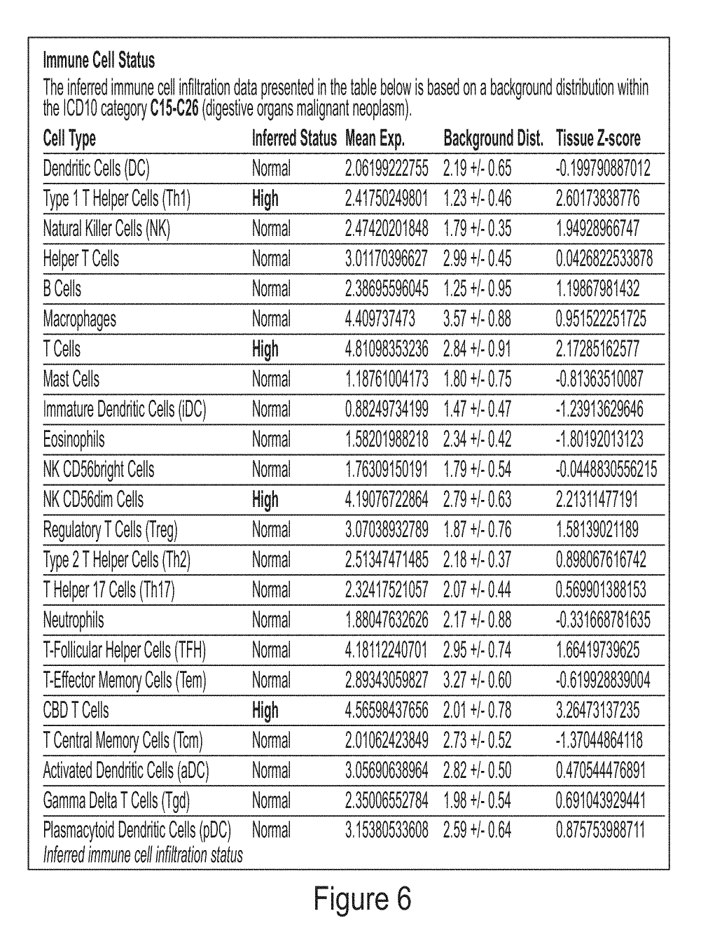

For example, a tumor tissue belonging to ICD10 class C15-C26 (here: digestive organs malignant neoplasm) can be analyzed using RNAseq and gene expression data quantified, using the specific tumor tissue type and the tabulated results of FIG. 5. Based on these results, as is exemplarily shown in FIG. 6, immune cell type status/presence can be readily inferred. In the example of FIG. 6, the tumor sample has higher than normal activity of Th1 cells, T cells, NK cd56dim cells, and CDB T cells. Viewed from a different perspective, it should be recognized that gene expression quantification of specific genes associated with specific immune cells (normalized by tumor tissue type) can be used to infer immune cell infiltration and/or immune cell activation. In this exemplary report format, an inferred status is included that indicates the kind and/or number of types of immune-cell types are elevated (e.g., 4 elevated signatures).

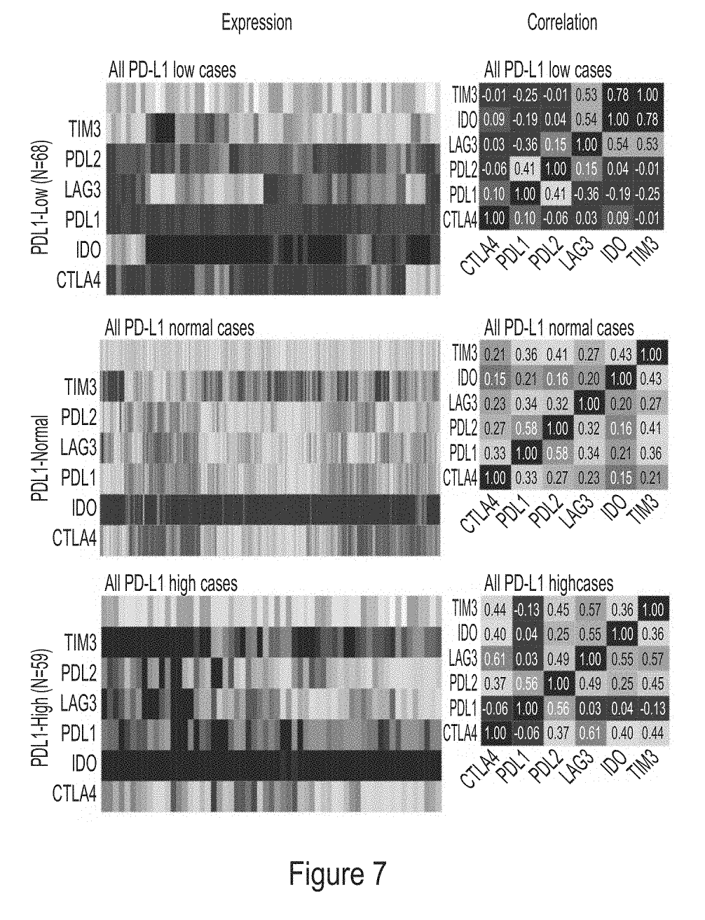

In still further studies, the inventor investigated whether or not immune marker co-expression patterns could be identified, and particularly checkpoint expression patterns and their correlations. For example, the inventors investigated if for a given PDL1 expression level in a tumor as measured by RNAseq any association could be identified with respect to other checkpoint related genes and their expression levels. More specifically, FIG. 7 shows exemplary checkpoint expression patterns. Here, the expression heatmaps are log2[tpm+1] scale with (blue=0, red>=5), and the colors at the top indicate the different ICD10 cancer types. Expression heatmaps are ordered by Euclidean distance, and the correlation plots are Pearson correlations (blue=0, red>=0.75). Notably, and in contrast to the immune gene expression patters discussed above, there was an apparent lack of significant tissue-dependent expression of immune checkpoints as can be taken from the unclustered appearance of the cancer type color indicators. As expected, however, PDL1 and PDL2 expression was moderately correlated.

Additionally, it was also observed that IDO and TIM3 had relatively high expression, particularly in the absence of PDL1 or in cases with low PDL1 expression. Expression levels of IDO and TIM3 were also highly correlated (R=0.78) when PDL1 is under-expressed, and that relationship seemed to be inversely proportional to PDL1 expression. LAG3 was also correlated with IDO and TIM3 in a low PDL1 setting, however, this relationship was not clear as PDL1 increased. Consequently, the data suggest that PDL1 itself is sufficient as a primary driver of immune suppression (as seen in the PDL1-high correlation plot), however when PDL1 is low there may be some differential role for IDO and TIM3.

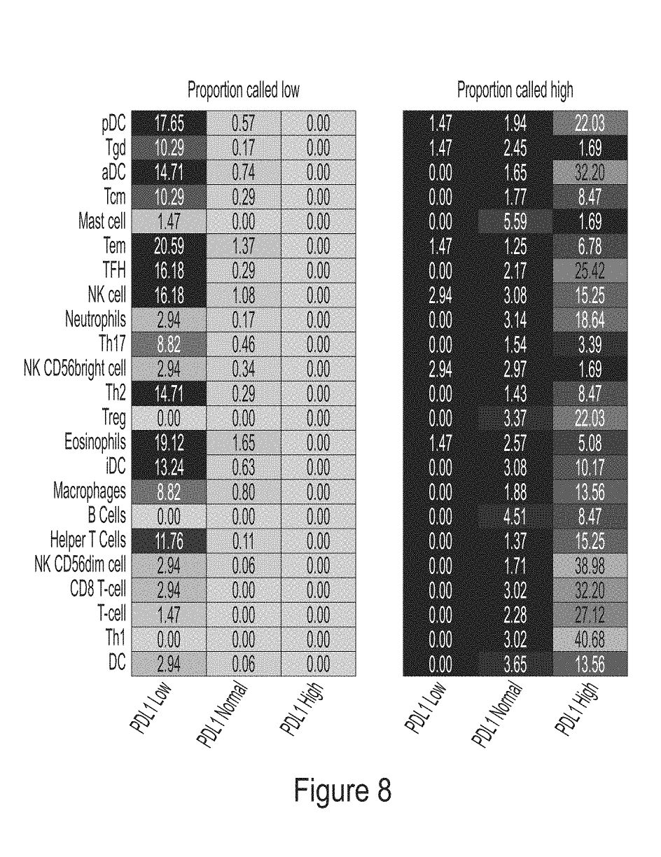

When further investigating the role of PDL1 with respect to immune cell categories as noted above, the inventor discovered that the PDL1 high group is enriched for multiple immune-cell types, including multiple kinds of T-cells & T-helper cells as can be seen in FIG. 8, right plot (depicting relative over-representation). Thus, especially in conjunction with the results of the checkpoint expression patterns seen in FIG. 7, it appears that PDL1 expression is probably sufficient to evade these systems. On the other hand, in the PDL1 low group CD8 T-Cells, T-Cells, and Th1 cells are not significantly under-represented, however most other category of immune cells are including NK, and memory T cells as can be seen in FIG. 8, left plot (depicting relative under-representation). Taken with the results of FIG. 7, the expression data are indicative that IDO and TIM3 have a strong role in regulating memory T cells.

Therefore, it should be appreciated that immune cell specific gene expression analysis can be used in predictive analysis of immune therapy, particularly for immune therapy targeting the PD1/PDL1 axis. On the other hand, alternative immune therapy targeting IDO and/or TIM3 may also be indicated where the tumor tissue is PDL1 low.

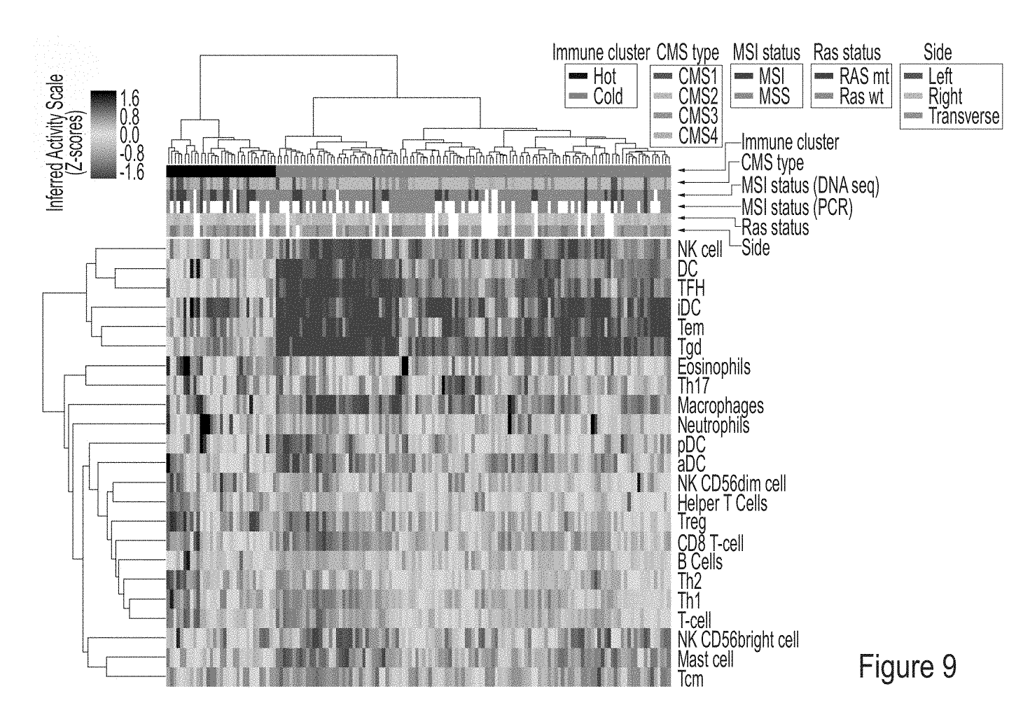

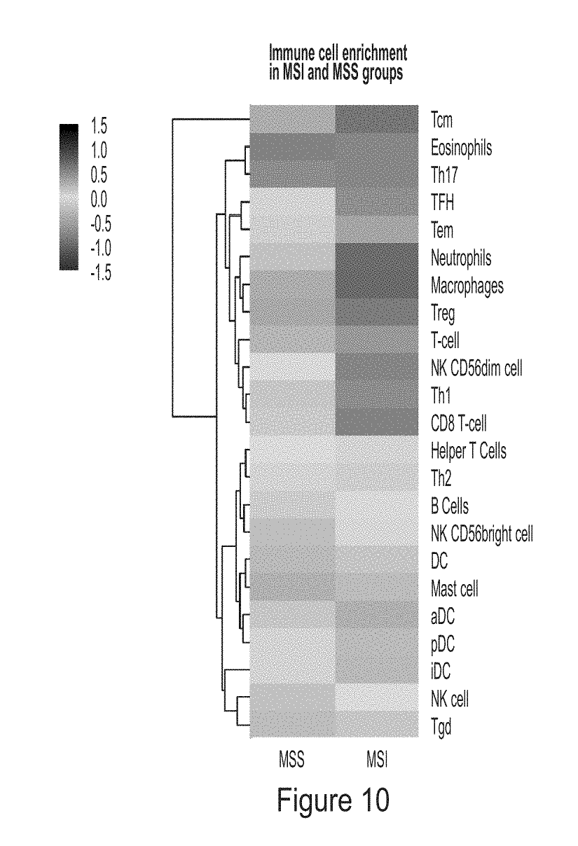

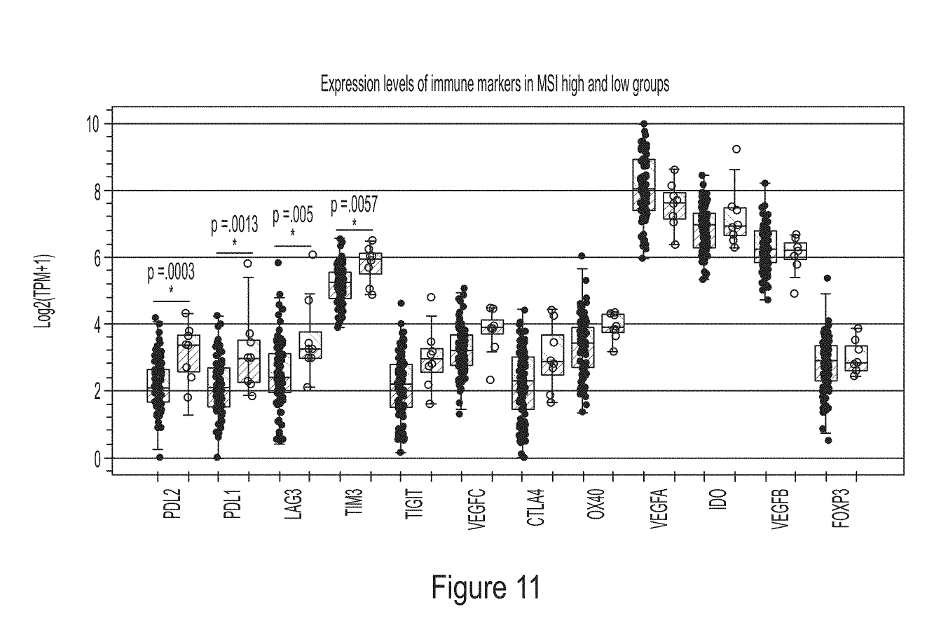

In still further experiments, immune status was also correlated with MSI status on a total of 152 colorectal cancer tumor samples. Tumor/normal-paired DNAseq (WGS or WES) and deep RNAseq was performed and MSI-status was determined by both PCR and WGS/WES profiles. CMS types, checkpoint expression, and immune-infiltration deconvolution were calculated upon RNAseq data using above sequences, and significant enrichment for MSI, immune status, CMS types, and clinical covariates were analyzed. FIG. 9 depicts exemplary results for the analysis. As can be seen from FIG. 9, clustering of immune expression bifurcated well in to hot and cold tumors. Moreover, significant association was found between CMS1, MSI, transverse sides, and being immunologically hot. Conversely, CMS2 was found to be significantly MSS, left-sided, and immunologically cold. Thus, CMS1 tumors that are immunologically hot appear to be treatable with immune checkpoint inhibitors. In yet another set of experiments, total of 521 GI patients with deep whole exome sequencing (WES) of tumor and blood samples, and whole transcriptomic sequencing (RNA-Seq) (˜200M reads per tumor) were available for this analysis from a commercial database. Variant calling was performed through joint probabilistic analysis of tumor and normal DNA reads, with germline status of variants being determined by heterozygous or homozygous alternate allele fraction in the germline sample. MSI was determined via a CLIA LDT based on NGS data at microsatellite sites. Notably, higher immune signaling was observed in MSI high tumors, and some MSI samples showed high CD8 T-cells enrichment. Moreover, and as observed before, TIM3 and LAG3 were expressed at higher levels in MSI high samples. Typical results are depicted in FIGS. 10 and 11. As can be seen from FIG. 10, enrichment of various immune cell types in the two MSI groups is shown. The brighter the red color is the larger the enrichment. Likewise, FIG. 11 illustrates the expression levels of various immune markers in the two MSI groups. Here, PDL2, PDL1, LAG3, and TIM3 are statistically significantly differentially expressed. TIM3 presents an interesting potential therapeutic target.

It should still further be appreciated that contemplated methods and analyses may also be useful in determination of suitable treatment where location may provide a contributing factor. For example, the inventor discovered that upper and lower GI tumors are distinct in their tolerated immune cell infiltration. Immune therapies should therefore be tailored based on location to take advantage of the innate immune apparatus present. Specifically, upper GI cancers appear especially fit for checkpoint therapy despite having lower average TMB.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

Moreover, all methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of characterizing a tumor, comprising:
quantifying or obtaining expression levels for a plurality of distinct genes, wherein the distinct genes are associated with respective distinct types of immune cells;
determining over-expression or under-expression for each of the distinct genes relative to respective reference ranges, wherein the reference ranges are specific for a specific tumor type; and
using the over-expression and/or under-expression of each of the distinct genes to infer activity and/or infiltration by the immune cells in the tumor.

2. The method of claim 1 wherein the expression level is measured via qPCR or RNAseq.

3. The method of claim 1 wherein the plurality of distinct genes is selected from the group consisting of BLK, CD19, CR2 (CD21 ), HLA-DOB, MS4A 1 (CD20), TNFRSF17 (CD269), CD2,CD3E,CD3G,CD6, ANP32B (APRIL), BATF, NUP107, CD28, ICOS (CD278), CD38, CSF2 (GM-CSF), IFNG, IL12B2,LTA, CTLA4 (CD152), TXB21, STAT4, CXCR6 (CD186), GATA3, IL26, LAIR2 (CD306), PMCH, SMAD2, STATE, IL 17A, IL 17RA (CD217), RORC, CXCL13, MAF, PDCD1 (CD279), BCL6, FOXP3, ATM, DOCKS, NEFL, REPS1, USP9Y, AKT3, CCR2 (CD192), EWSR1 (EWS), LTK, NFATC4, CD8A, CD8B, FLT3LG, GZMM, MET1, PRF1, CD160, FEZ1, TARP (TCRG), BCL2, FUT5, NCR1 (CD335), ZNF205, FOXJ1, MPPED1, PLA2G6, RRAD, GTF3C1, GZMB, IL21R (CD360), CCL13, CCL17, CCL22 (MDC), CD209, HSD11B1, CD1A, CD1B, CD1E, F13A1, SYT17, CCL1, EBI3, IDO1 (INDO), LAMP3 (CD208), OAS3, IL3RA (CD123), APOE, CCL 7 (FIC), CD68, CHIT1, CXCL5, MARCO, MSR1 (CD204), CMA1, CTSG, KIT (CD117), MS4A2, PRG2, TPSAB1, CSF3R (CD114), FPR2, MME (CD10), CCR3 (CD193), IL5RA (CD125), PTGDR2, (CD294), SMPD3, and THBS1.

4. The method of claim 1 wherein the over-expression or under-expression is determined when the quantified expression level exceeds +/−2SD of the reference range.

5. The method of claim 1 wherein the reference ranges are specific for a specific tumor type as classified in ICD10.

6. The method of claim 4 wherein the reference ranges are specific for a specific tumor type as classified in ICD10.

7. The method of claim 1 further comprising a step of associating an immune status with the tumor based on the inferred activity and/or infiltration.

8. The method of claim 1 further comprising a step of recommending a treatment with a checkpoint inhibitor.

9. The method of claim 7 wherein the checkpoint inhibitor is a PDL1 inhibitor for a PDL1-high tumor.

10. The method of claim 7 wherein the checkpoint inhibitor is a TIM3 inhibitor or an IDO inhibitor for a PDL1-low tumor.

11. A method of identifying a patient for immune therapy of a tumor, comprising:
quantifying or obtaining expression levels for a plurality of distinct genes, wherein the distinct genes are associated with respective distinct types of immune cells;
determining over-expression or under-expression for each of the distinct genes relative to respective reference ranges, wherein the reference ranges are specific for a specific tumor type;
using the over-expression and/or under-expression of each of the distinct genes to infer activity and/or infiltration by the immune cells in the tumor; and
using the inferred activity to predict an increased likelihood of positive treatment outcome where the inferred activity and/or infiltration of distinct immune cells in the tumor is increased relative to the respective reference ranges; and
identifying the patient for immune therapy upon prediction of the increased likelihood.

12. The method of claim 11 wherein the distinct immune cells in the tumor are selected from pDC, aDC, TFH, NK cells, neutrophils, Treg, iDC, macrophages,Thelper cells, NK cells, CD8 T cells, T cells, and Th1 cells.

13. The method of claim 11 wherein the increased number is observed in at least two distinct immune cells in the tumor.

14. The method of claim 11 wherein the increased number is observed in at least four distinct immune cells in the tumor.

15. The method of claim 11 wherein the plurality of distinct genes is selected from the group consisting of BLK, CD19, CR2 (CD21 ), HLA-DOB, MS4A 1 (CD20), TNFRSF17 (CD269), CD2,CD3E,CD3G,CD6, ANP32B (APRIL), BATF, NUP107, CD28, ICOS (CD278), CD38, CSF2 (GM-CSF), IFNG, IL12B2,LTA, CTLA4 (CD152), TXB21, STAT4, CXCR6 (CD186), GATA3, IL26, LAIR2 (CD306), PMCH, SMAD2, STATE, IL 17A, IL 17RA (CD217), RORC, CXCL13, MAF, PDCD1 (CD279), BCL6, FOXP3, ATM, DOCKS, NEFL, REPS1, USP9Y, AKT3, CCR2 (CD192), EWSR1 (EWS), LTK, NFATC4, CD8A, CD8B, FLT3LG, GZMM, MET1, PRF1, CD160, FEZ1, TARP (TCRG), BCL2, FUTS, NCR1 (CD335), ZNF205, FOXJ1, MPPED1, PLA2G6, RRAD, GTF3C1, GZMB, IL21R (CD360), CCL13, CCL17, CCL22 (MDC), CD209, HSD11B1, CD1A, CD1B, CD1E, F13A1, SYT17, CCL1, EBI3, IDO1 (INDO), LAMP3 (CD208), OAS3, IL3RA (CD123), APOE, CCL 7 (FIC), CD68, CHIT1, CXCL5, MARCO, MSR1 (CD204), CMA1, CTSG, KIT (CD117), MS4A2, PRG2, TPSAB1, CSF3R (CD114), FPR2, MME (CD10), CCR3 (CD193), IL5RA (CD125), PTGDR2, (CD294), SMPD3, and THBS1.

16. The method of claim 11 wherein the over-expression or under-expression is determined when the quantified expression level exceeds +/−2SD of the reference range.

17. The method of claim 11 wherein the immune therapy comprises treatment with at least a checkpoint inhibitor.

18. The method of claim 11 wherein the immune therapy comprises treatment with at least one of a vaccine composition and an immune stimulatory cytokine.

19. The method of claim 11 further comprising a step of determining expression of at least one checkpoint related gene.

20. The method of claim 11 further comprising a step of determining CMS class or MSI status.
